(19) United States
(12) Patent Application Publication
(10) Pub. No.: US 2014/0273200 A1
(43) Pub. Date: Sep. 18, 2014

(54) Apparatus for optimizing temperature for fermentation process inside of a variety of enclosures.

(71) Applicants: Thomas Rozek, (US); Iva Rozek, (US)

(72) Inventors: Thomas Rozek, (US); Iva Rozek, (US)

(21) Appl. No.: 13/845,019

(22) Filed: Mar. 17, 2013

(51) Int. Cl. C12M 1/02

(52) U.S. Cl. CPC C12M 41/22
USPC 435/303.1

(57) ABSTRACT

An apparatus designed to be placed inside of variety of enclosures to control the temperature inside of the enclosure to create optimal fermentation conditions, consisting of: heater, controller, temperature sensor and optionally a fan. The apparatus fits inside of an independent enclosures of various sizes and types, typically thermally insulated such as existing coolers. Such apparatus placed inside an enclosure can create optimal temperature inside the enclosure for fermentation of various yeast and bacteria as would be used for example in preparation of foods such as yogurt, kimchi, kefir and proofing of baked goods.

### CROSS-REFERENCE TO RELATED APPLICATION

This application claims priority to the U.S. Provisional Patent Application No. 61/612,352, filed on Mar. 18, 2012, which is incorporated herein in its entirety.

### FIELD OF THE INVENTION

The present invention relates to optimizing temperature of yeast or bacterial culture with a small apparatus inside of an enclosure. The enclosure is selected by user for its convenience. The convenience may be by the fact that the user already owns such enclosure or that a given enclosure is suitable size and shape for the containers that the user wants to use to hold the fermentation mixture.

Fermentation uses bacteria or yeast cultures to convert organic compounds. Temperature plays an extremely vital role in the fermentation process as different yeasts and bacteria thrive at different temperatures and are dormant at others. By controlling temperature of fermentation it is possible to stimulate desired yeast and/or bacteria activity and suppress the activity of other undesired yeast and/or bacteria.

For example yogurt is prepared by the fermentation of bacteria (Lactobacillus Bulgaricus and Streptococcus thermophilus or other combinations) mixed with milk at the temperature of around 42° C. The texture of yogurt can be effected by fermenting at different temperatures. When preparing baked goods, yeast fermentation is encouraged during dough proofing by keeping temperature of the dough at about 40° C. to increase and speed up dough rise.

### DESCRIPTION OF PRIOR ART

There are existing devices designed for controlling the temperature of a fermentation process. The devices have a space or container(s) for the fermentation mixture inside of en enclosure. These devices have heaters, controls and thermostats that are integrated into the enclosure or have parts (such as the temperature controller) outside of the enclosure.

Examples of flaws with the current devices:
Existing home yogurt and kefir makers come with specific fermentation containers that limit the volume of fermenting mixture.
Some existing home yogurt makers come with small fermentation containers that are not suitable for preparation of speciality yogurts that requires further processing after the fermentation (for example Greek yogurt), which means the content of the small containers has to be emptied into one large container for the further processing.
Existing bread proofers, yogurt and kefir makers are large to store.

### SUMMARY OF INVENTION

The purpose of this invention is to provide a convenient small device that provides flexibility to a user as to what containers to use for holding fermentation mixture, and the amount of fermentation mixture processed.

The novelty of this apparatus consists in the fact that it is independent of the enclosure that creates the volume used to hold the containers with fermentation mixture. By being independent users can employ coolers or other enclosures they may already own for their fermentation needs. The apparatus may be used in different enclosures depending on the fermentation volume required. The same apparatus may be employed to perform different fermentation such as yogurt preparation and bread proofing with a single device.

### BRIEF DESCRIPTION OF THE DRAWING

FIG. 1 is a schematic view of the proposed invention inside an enclosure such as a cooler.

### DESCRIPTION OF PREFERRED EMBODIMENT

Referring to FIG. 1, the apparatus operates inside of an enclosure 1 that provides thermal insulation from ambient air surrounding the enclosure and creates the fermentation compartment. The apparatus and container(s) holding fermentation mixture are place inside the enclosure 1.

The apparatus housed in housing 3, operates by heating the fermentation compartment air with electrical heater 7. The temperature inside of the fermentation compartment is generally higher than the ambient temperature outside of the enclosure 1.

By heating the fermentation container air that circulates as shown by arrow 8 the air temperature inside the fermentation compartment 1, can be maintained for optimal fermentation of the mixture inside one or more containers 2. The heat may be circulated by natural convection or assisted with fan 6 causing forced convection. The heat may also be transferred by conduction to the fermentation mixture.

The fermentation compartment temperature is monitored with temperature sensor 5 that is used by controller 4 to control the amount of power released by heater 7 and optionally to control the operation of Fan 6.

The controller 4 can incorporate display, manual controls, wired or wireless interface, and/or other sensors to allow programming and monitoring of the fermentation conditions such as temperature, temperature changes and durations.

What is claimed:

1. A fermentation apparatus that is placed inside of an enclosure that heats the inside of said enclosure to a temperature optimized for fermentation of a mixture(s) in one or more container(s) that are also placed inside said enclosure, the apparatus comprising: Temperature controlled heater, housing.

2. A fermentation apparatus that is placed inside of an enclosure that heats the inside of said enclosure to a temperature optimized for fermentation of a mixture(s) in one or more container(s) that are also placed inside said enclosure, utilizes forced convection heating with the use of a fan, the apparatus comprising: Temperature controlled heater, housing, fan.

3. A fermentation apparatus that is placed inside of an enclosure that heats the inside of said enclosure to a temperature optimized for fermentation of a mixture(s) in one or more container(s) that are also placed inside said enclosure, utilizing conduction heating with the use of heat conducting material structure between the heater and the fermentation mixture container(s), the apparatus comprising: Temperature controlled heater, housing, thermally conductive structure.

4. A fermentation apparatus described in claim 1 where said temperature controlled heater is adjustable to adjust the temperature of the fermentation compartment.

5. A fermentation apparatus described in claim 2 where said temperature controlled heater is adjustable to adjust the temperature of the fermentation compartment.

6. A fermentation apparatus described in claim 3 where said temperature controlled heater is adjustable to adjust the temperature of the fermentation compartment.
